(19) United States
(12) Patent Application Publication
(10) Pub. No.: US 2009/0117039 A1
(43) Pub. Date: May 7, 2009

(54) CHARGED BIODEGRADABLE POLYMERS FOR MEDICAL APPLICATIONS

(75) Inventor: Robert E. Richard, Wrentham, MA (US)

Correspondence Address: MAYER & WILLIAMS PC, 251 NORTH AVENUE WEST, 2ND FLOOR, WESTFIELD, NJ, 07090, US

(73) Assignee: BOSTON SCIENTIFIC SCIMED, INC., Maple Grove, MN (US)

(21) Appl. No.: 12/263,167

(22) Filed: Oct. 31, 2008

Related U.S. Application Data
(60) Provisional application No. 61/001,538, filed on Nov. 2, 2007.

(51) Int. Cl. A61K 9/14; A61K 51/12; A61K 51/04; A61F 2/82; A61P 9/00

(52) U.S. Cl. 424/1.61; 424/501; 424/1.11; 424/1.65; 424/423

(57) ABSTRACT

In accordance with one aspect of the invention, implantable medical devices are provided which include polymeric compositions that comprise at least one type of biodegradable ionic polymer. The at least one type of biodegradable ionic polymer comprises a biodegradable polymer core and one or more ionic end groups.

### STATEMENT OF RELATED APPLICATIONS

This application claims the benefit of U.S. Provisional Patent Application Ser. No. 61/001,538, filed Nov. 2, 2007, entitled “Charged Biodegradable Polymers For Medical Applications”, which is incorporated by reference herein in its entirety.

### FIELD OF THE INVENTION

The invention relates to charged biodegradable polymers for medical applications, including the use of the same in injectable particles.

### BACKGROUND OF THE INVENTION

Many clinical situations benefit from regulation of the vascular, lymphatic or duct systems by restricting the flow of body fluid or secretions. For example, the technique of embolization involves the therapeutic introduction of particles into the circulation to occlude blood vessels. Permanent or temporary occlusion of blood vessels is desirable for managing various diseases, disorders and conditions. For example, permanent or temporary occlusion of blood vessels can be used to either arrest or prevent hemorrhaging or to cut off blood flow to a structure or organ.

Various polymer-based microspheres are currently employed to embolize blood vessels. These microspheres are usually introduced to the location of the intended embolization through microcatheters. Current commercially available embolic microspheres are composed of biostable polymers. Materials commonly used commercially for this purpose include polyvinyl alcohol (PVA), acetalized PVA (e.g., Contour® embolic agent, Boston Scientific, Natick, Mass., USA) and crosslinked acrylic hydrogels (e.g., Embospheres®, Biosphere Medical, Rockland, Mass., USA). Similar devices have been used in chemoembolization to increase the residence time of a therapeutic agent after delivery. In one specific instance, a therapeutic agent (doxorubicin) has been directly added to hydrogel microspheres (prepared from N-acrylamidoacetaldehyde derivatized polyvinyl alcohol copolymerized with 2-acrylamido-2-methylpropane sulfonate) such that the therapeutic agent can be released locally after delivery (e.g., DC Bead™ drug delivery chemoembolization system, Biocompatibles International plc, Farnham, Surrey, UK). Other examples of commercially available microspheres include glass microspheres with entrapped radioisotopes (e.g., ⁹⁰Y), in particular, TheraSpheres™, MDS Nordion, Ottowa, Canada and polymer microspheres that contain monomers that are capable of chelating radioisotopes (e.g., ⁹⁰Y), in particular, SIR-Spheres®, SIRTex Medical, New South Wales, Australia.

It is also known to use polymer-based microspheres as augmentative materials for aesthetic improvement, including improvement of skin contour. Furthermore, polymer-based microspheres have also been used as augmentative materials in the treatment of various diseases, disorders and conditions, including urinary incontinence, vesicourethral reflux, fecal incontinence, intrinsic sphincter deficiency (ISD) and gastro-esophageal reflux disease. For instance, a common method for treating patients with urinary incontinence is via periurethral or transperineal injection of a bulking agent that contains polymer-based microspheres. The bulking agent is injected into a plurality of locations, assisted by visual aids, causing the urethral lining to coapt.

### SUMMARY OF THE INVENTION

In accordance with one aspect of the invention, implantable medical devices are provided which include polymeric compositions that comprise at least one type of biodegradable ionic polymer. The at least one type of biodegradable ionic polymer comprises a biodegradable polymer core and one or more ionic end groups.

These and various additional aspects, as well as various embodiments and advantages of the present invention will become immediately apparent to those of ordinary skill in the art upon review of the Detailed Description and any Claims to follow.

### DETAILED DESCRIPTION

In accordance with one aspect of the invention, implantable medical devices are provided which include polymeric compositions that comprise at least one type of biodegradable ionic polymer. The at least one type of biodegradable ionic polymer comprises a biodegradable polymer core and one or more ionic end groups.

Biodegradable ionic polymers for the practice of the invention may have cationic end groups, anionic end groups, or both. Moreover, polymeric compositions in accordance with the present invention may contain a mixture of (a) ionic polymers with anionic end groups and (b) ionic polymers with cationic end groups. The addition of ionic end groups to the biodegradable polymer cores provides “ionomer” type properties which lead to the formation of crosslinks within the polymer which can impart elasticity to polymer compositions formed from the same when in an aqueous environment. The addition of ionic groups also may enhance water swellability (e.g., swellability in bodily fluids) to polymeric compositions formed from such polymers. For example, where the polymeric compositions are embolic particles, the swelling characteristic of the particles may improve the embolization efficiency of the particles (e.g., in vivo swelling may lead to an increased embolization effect). Moreover, the swelling characteristics of polymeric compositions containing therapeutic agents may be adjusted to modulate therapeutic agent release (e.g., increased in vivo swelling may result in increased therapeutic agent release and vice versa). Ionic groups may also impart the potential to bind therapeutic agents based on electrostatic interactions (e.g., charge-charge interactions, charge-dipole interactions, etc.), retarding release of the same.

Medical devices in accordance with the invention may be used to treat various diseases and conditions in a variety of subjects. Subjects include vertebrate subjects, particularly humans and various warm-blooded animals, including pets and livestock. As used herein, “treatment” refers to the prevention of a disease or condition, the reduction or elimination of symptoms associated with a disease or condition, or the substantial or complete elimination of a disease or condition.

As used herein a “polymeric composition” (e.g., polymeric particle, polymeric coating, etc.) is one that contains polymers, for example, from 50 wt % or less to 75 wt % to 90 wt % to 95 wt % to 97.5 wt % to 99 wt % or more polymers (on a dry weight basis).

As used herein, “polymers” are molecules that contain multiple copies of one or more types of constitutional units, commonly referred to as monomers. The number of monomers/constitutional units within a given polymer may vary widely, ranging, for example, from 5 to 10 to 25 to 50 to 100 to 1000 to 10,000 or more constitutional units. As used herein, the term “monomers” may refer to free monomers and to those that are incorporated into polymers, with the distinction being clear from the context in which the term is used.

As noted above, biodegradable ionic polymers (also referred to herein as “ionic polymers” or “ionomers”) in accordance with the invention comprise a biodegradable polymer core and one or more ionic end groups.

Biodegradable polymer cores for use in the biodegradable ionic polymers of the present invention can have a variety of architectures, linear and branched architectures. Branched architectures include star-shaped architectures (e.g., architectures in which three or more chains emanate from a single branch point), comb architectures (e.g., architectures having a main chain and a plurality of side chains, such as graft polymers) and dendritic architectures (e.g., arborescent and hyperbranched polymers), among others.

Polymers containing a single type of monomer may be referred to herein as homopolymers, whereas polymers containing two or more types of monomers may be referred to herein as copolymers. The two or more types of monomers within a given copolymer may be present in any of a variety of distributions including random, statistical, gradient and periodic (e.g., alternating) distributions, among others. One particular type of copolymer is a “block copolymer,” which is a copolymer that contains two or more polymer blocks of different composition. As used herein, a “block” or “polymer block” is a grouping of constitutional units (e.g., 5 to 10 to 25 to 50 to 100 to 250 to 500 to 1000 or more units). Blocks can be unbranched or branched. Blocks can contain a single type of constitutional unit (also referred to herein as “homopolymeric blocks”) or multiple types of constitutional units (also referred to herein as “copolymeric blocks”) which may be present, for example, in a random, statistical, gradient, or periodic (e.g., alternating) distribution. As used herein, a “polymer chain” is linear polymer block.

As used herein, a polymer or polymer core is “biodegradable” if it undergoes bond cleavage in vivo, regardless of the mechanism of bond cleavage (e.g., enzymatic breakdown, hydrolysis, oxidation, etc.).

Polymeric compositions in accordance with the invention are bioresorbable. As used herein, a polymeric composition is “bioresorbable” if it disintegrates in vivo due to one or more mechanisms such as dissolution, biodegradation, and so forth.

In many embodiments, the polymeric compositions in accordance with the invention are swellable. For example, when in a dry state, polymeric compositions in accordance with the invention may swell by at least 10% in water, for instance, swelling by 10% to 25% to 50% to 100% to 200% to 500% or more. Swelling of a composition may be characterized by the following formula: % swelling=((mₜ−m₀)/m₀)×100, where m₀ is the original composition weight and mₜ is the composition weight at time t (e.g., evaluated at 1 hr, 4 hrs, 24 hrs, etc.).

Various exemplary embodiments of the invention will now be described that pertain to injectable particles. However, the invention is not so limited. Additional embodiments include, for example, the use of the above described biodegradable ionic polymers in bulking agents and tissue engineering scaffolds, as well as in polymeric coatings for implantable medical device substrates, including polymeric coatings for metallic (e.g., stainless steel, nitinol, etc.) vascular stents, among other devices. The polymeric stent coatings may optionally further include one or more additional agents, including anti-restenotic agents.

With respect to injectable particles in accordance with the invention, such particles may vary widely in shape. In certain embodiments, they are substantially spherical, for example, having the form of a perfect (to the eye) sphere or the form of a near-perfect sphere such as a prolate spheroid (a slightly elongated sphere) or an oblate spheroid (a slightly flattened sphere), among other possibilities. In other embodiments they may be in the form of another regular geometry (e.g., cylindrical, etc.) or an irregular geometry. In embodiments where the particles are substantially spherical, at least half of the particles (50% or more, for example, from 50% to 75% to 90% to 95% or more of a particle sample) may have a sphericity of 0.8 or more (e.g., from 0.80 to 0.85 to 0.9 to 0.95 to 0.97 or more). The sphericity of a collection of particles can be determined, for example, using a Beckman Coulter RapidVUE Image Analyzer version 2.06 (Beckman Coulter, Miami, Fla.). Briefly, the RapidVUE takes an image of continuous-tone (gray-scale) form and converts it to a digital form through the process of sampling and quantization. The system software identifies and measures the particles in an image. The sphericity of a particle, which is computed as Da/Dp (where Da=√(4A/π); Dp=P/π; A=pixel area; P=pixel perimeter), is a value from zero to one, with one representing a perfect circle.

The injectable particles of the invention can vary in size, with typical longest linear cross-sectional dimensions (e.g., for a sphere, the diameter) ranging, for example, from 40 to 100 to 150 to 250 to 500 to 750 to 1000 to 1500 to 2000 to 2500 to 5000 microns (μm).

For a collection of particles, the arithmetic mean maximum for the group typically ranges, for example, from 40 to 100 to 150 to 250 to 500 to 750 to 1000 to 1500 to 2000 to 2500 to 5000 microns (μm). The arithmetic mean maximum dimension of a group of particles can be determined using a Beckman Coulter RapidVUE Image Analyzer version 2.06 (Beckman Coulter, Miami, Fla.), described above. The arithmetic mean maximum dimension of a group of particles (e.g., in a composition) can be determined by dividing the sum of the diameters (or the longest dimension for non-spherical/irregular particles) of all of the particles in the group by the number of particles in the group.

In some embodiments, at least 95 vol % of the particles within a group have longest linear cross-sectional dimensions between 40 μm and 5000 μm. For example, where the particles are spherical at least 95 vol % of the particles may have diameters between 40 μm and 5000 μm. More particularly, depending on the embodiment, at least 95 vol % of the particles within a group may have longest linear cross-sectional dimensions between any two of the following dimensions: 40, 100, 150, 250, 500, 750, 1000, 1500, 2000, 2500 and 5000 microns.

In some embodiments, the particles are porous particles. As used herein a “porous particle” is a particle that contains pores, which may be observed, for example, by viewing the pores using a suitable microscopy technique such as scanning electron microscopy. Pore size may vary widely, ranging from 1 micron or less to 2 microns to 5 microns to 10 microns to 25 microns to 50 microns to 100 microns or more. Pores can come in a wide range of shapes. Pores can be connected (open cell) or discrete (closed cell).

As noted above polymeric compositions in accordance with the present invention (e.g., particles, coatings, etc.) are formed using biodegradable ionic polymers that comprise a biodegradable polymer core and one or more ionic end groups. Such polymers may contain cationic end groups, anionic end groups, or both. In certain embodiments, ionic groups are found only at the chain ends of the polymer core, rather than within the polymer core (e.g., rather than along the backbone of a polymer chain or chains within the polymeric core).

Typically, the number average molecular weight of the biodegradable ionic polymers of the invention ranges, for example, from 1000 to 100,000 kDa or more.

As indicated above, the biodegradable polymer cores of the ionic polymers of the invention can have a variety of polymer architectures, including linear and branched architectures (e.g., star-shaped architectures, comb architectures, dendritic architectures, etc.), among others. Because the ionic polymers have ionic end groups, for polymers of comparable molecular weight, ionic polymers with more highly branched biodegradable polymer cores generally have higher charge densities (because they have more chain ends) than ionic polymers having less highly branched biodegradable polymer cores.

A wide variety of cationic and anionic end groups can be employed in the ionic polymers of the invention. Examples of cationic end groups include cations based on the following proton accepting end groups (with cations in parentheses): —NH₂ (cation: —NH₃⁺), —NHR₁ (cation: —NH₂R₁⁺) and —NR₁R₂ (cation: —NHR₁R₂⁺), as well as —NR₁R₂ R₃⁺ end groups, among others, where R₁, R₂ and R₃ are independently C1-C10 linear or branched alkyl groups. Examples of cationic end groups further include phosponium cations such as —PH₃⁺, —PH₂R₁⁻, —PHR₁R₂⁺ and —PR₁R₂R₃⁺. Examples of anionic end groups include anions based on the following proton donating end groups (with anions in parenthesis): —COOH (anion: —COO^{'}), —SO₃H (anion: —SO₃⁻), —OSO₃H (anion: —OSO₃⁻), —PO(OH)₂ (anions: —PO₂(OH)⁻ and —PO₃²⁻), and —OPO(OH)₂ (anions: —OPO₂(OH)⁻ and —OPO₃²⁻), among others.

The ionic polymers of the invention can be based on a wide variety of biodegradable polymer cores. In this regard, ionic polymers for use in the invention may be formed, for example, from essentially any biodegradable polymer core with end groups capable of being converted into ionic end groups. Specific examples of biodegradable polymer cores may be selected from those consisting of or containing one or more polymer blocks (e.g., one or more polymer chains) selected from the following, among many others: (a) polyester homopolymers and copolymers such as polyglycolic acid (PGA), polylactic acid (PLA) including poly-L-lactic acid, poly-D-lactic acid and poly-D,L-lactic acid, poly(beta-hydroxybutyrate), polygluconate including poly-D-gluconate, poly-L-gluconate, poly-D,L-gluconate, poly(epsilon-caprolactone), poly(delta-valerolactone), poly(p-dioxanone), poly(lactic acid-co-glycolic acid) (PLGA), poly(lactic acid-co-delta-valerolactone), poly(lactic acid-co-epsilon-caprolactone), poly(lactic acid-co-beta-malic acid), poly(beta-hydroxybutyrate-co-beta-hydroxyvalerate), poly[1,3-bis(p-carboxyphenoxy)propane-co-sebacic acid], and poly(sebacic acid-co-fumaric acid), among others, (b) polycarbonate homopolymers and copolymers such as poly(trimethylene carbonate), poly(lactic acid-co-trimethylene carbonate) and poly(glycolic acid-co-trimethylene carbonate), among others, (c) poly(ortho ester) homopolymers and copolymers such as those synthesized by copolymerization of various diketene acetals and diols, among others, (d) polyanhydride homopolymers and copolymers such as poly(adipic anhydride), poly(suberic anhydride), poly(sebacic anhydride), poly(dodecanedioic anhydride), poly(maleic anhydride), poly[1,3-bis(p-carboxyphenoxy)methane anhydride], and poly[alpha,omega-bis(p-carboxyphenoxy)alkane anhydrides] such as poly[1,3-bis(p-carboxyphenoxy)propane anhydride] and poly[1,3-bis(p-carboxyphenoxy)hexane anhydride], among others, and (e) amino-acid-based polymers including tyrosine-based polyarylates (e.g., copolymers of a diphenol and a diacid linked by ester bonds, with diphenols selected, for instance, from ethyl, butyl, hexyl, octyl and bezyl esters of desaminotyrosyl-tyrosine and diacids selected, for instance, from succinic, glutaric, adipic, suberic and sebacic acid), tyrosine-based polycarbonates (e.g., copolymers formed by the condensation polymerization of phosgene and a diphenol selected, for instance, from ethyl, butyl, hexyl, octyl and bezyl esters of desaminotyrosyl-tyrosine), and tyrosine-, leucine- and lysine-based polyester-amides; specific examples of tyrosine-based polymers include includes polymers that are comprised of a combination of desaminotyrosyl tyrosine hexyl ester, desaminotyrosyl tyrosine, and various di-acids, for example, succinic acid and adipic acid, among others.

One example of a process by which ionic biodegradable polymers can be produced has recently been reported by B. Atthoff et al., Macromolecules, 2006, 39 (11): 3907-3913 and B. Atthoff et al., Biomacromolecules, 7 (2006) 2401-2406. These authors describe ionic polymers having poly(trimethylene carbonate) (PTMC) polymer cores. The ionic polymers are formed by first synthesizing two PTMC chains using a 1,4-butane diol as an initiator. Then, the two hydroxyl end groups of the resulting polymer were converted into either cationic end groups (i.e., trimethyl ammonium groups) or anionic end groups (i.e., sulfonate groups). The ionic character of the resulting ionic polymers provides compositions that are formed from such ionic polymers with swellable properties, with reported swelling ranging from 37 to 590%, depending on the molecular weight to the PTMC polymer core, the nature of the end groups, and the nature of the aqueous solution that is used to induce the swelling. The ionic character also increases the elasticity of compositions formed from such polymers, which the authors hypothesize is a result of the formation of ionic phase domains in the bulk, which act as physical crosslinks for the PTMC. PTMC is said to ordinarily behave as an amorphous melt above the glass transition temperature (approx. −50° C.).

The 1,4-butane diol initiator employed by Atthoff et al. results in a two arm polymer in which two hydroxyl-terminated PTMC chains extend from a residue of the 1,4-butane diol. By employing a initiators with three or more (e.g., 3, 4, 5, 6, etc.) hydroxyl groups, branched polymers, specifically star polymers having a number of arms corresponding to the number of hydroxyl groups on the initiator (e.g., 3, 4, 5, 6, etc.) arms, may be created, with each arm terminating in a hydroxyl group. The hydroxyl groups may then be converted to cationic or anionic end groups, for example, using procedures like those described in Atthoff et al. Examples of hydroxyl terminated polymers include polymers containing hydroxyl terminated homopolymer and copolymer chains that may be selected from polycarbonate chains, polyester chains, and poly(ortho ester) chains.

In some embodiments, the polymeric compositions of the present invention may further contain one or more optional agents such as therapeutic agents, imaging agents, and so forth.

For example, injectable particles in accordance with the invention may further contain one or more therapeutic agents or they may be provided in a kit with one or more therapeutic agents that may be loaded into the particles in a clinical setting, among other options.

Among other characteristics, the optional therapeutic agents may be, for example, hydrophobic, hydrophilic or amphiphilic, and they may be negatively charged, positively charged, zwitterionc, or of neutral charge.

As noted above, being charged, biodegradable ionic polymers in accordance with the present invention are capable of binding certain therapeutic agents based on electrostatic interactions (e.g., charge-charge interactions, charge-dipole interactions, etc.), and they may delay release of therapeutic agents based on these interactions. For example, the ionic polymers may electrostatically bind therapeutic agents of opposite charge (e.g., based on charge-charge interactions), they may bind therapeutic agents via complexation (e.g., based on charge-dipole interactions), and so forth.

Examples of therapeutic agents for use in the compositions of the present invention include anti-thrombotic/anti-clotting/anti-coagulant agents (e.g., heparin, heparin derivatives, urokinase, dextrophenylalanine proline arginine chloromethylketone or “PPack”, RGD peptide-containing compounds, hirudin, anti-thrombin compounds including anti-thrombin antibodies, platelet receptor antagonists, anti-platelet receptor antibodies, aspirin, prostaglandin inhibitors, platelet inhibitors, tick antiplatelet factors or peptides, etc.); thrombogenic agents and agents that promote clotting; antioxidants; angiogenic agents, anti-angiogenic agents; anti-proliferative agents; calcium entry blockers (e.g., verapamil, diltiazem, nifedipine); survival genes which protect against cell death (e.g., anti-apoptotic Bcl-2 family factors and Akt kinase); steroidal and non-steroidal anti-inflammatory agents (e.g., dexamethasone, prednisolone, corticosterone, budesonide, estrogen, acetyl salicylic acid, sulfasalazine, mesalamine, etc.); protein kinase and tyrosine kinase inhibitors; cytostatic agents (i.e., agents that prevent or delay cell division in proliferating cells, for example, by inhibiting replication of DNA or by inhibiting spindle fiber formation) (e.g., toxins such as ricin toxin and radioisotopes, methotrexate, adriamycin, radionuclides, protein kinase inhibitors such as staurosporin and diindoloalkaloids, etc.), agents that inhibit intracellular increase in cell volume (i.e., the tissue volume occupied by a cell) such as cytoskeletal inhibitors (e.g., colchicine, vinblastin, cytochalasins, paclitaxel, etc.) or metabolic inhibitors (e.g., staurosporin, Pseudomonas exotoxin, modified diphtheria and ricin toxins, etc.); trichothecenes (e.g., a verrucarin or roridins); agents acting as inhibitors that block cellular protein synthesis and/or secretion or organization of extracellular matrix (i.e., an “anti-matrix agent” such as colchicine or tamoxifen); anti-restenotic agents (e.g., paclitaxel, olimus family drugs such as sirolimus, everolimus, tacrolimus, zotarolimus, etc.), various pharmaceutically acceptable salts and derivatives of the foregoing, and combinations of the foregoing, among other agents.

Examples of therapeutic agents which may be used in the compositions of the invention thus include various agents able to kill undesirable cells (e.g., those making up cancers and other tumors such as uterine fibroids) or to slow or arrest growth of undesirable cells, among other agents.

Further specific examples of therapeutic agents for use in the compositions of the invention, not necessarily exclusive of those above, may be selected from suitable members of the following: radioisotopes (e.g., ⁹⁰Y, ³²P, ¹⁸F, ¹⁴⁰La, ¹⁵³Sm, ¹⁶⁵Dy, ¹⁶⁶Ho, ¹⁶⁹Er, ¹⁶⁹Yb, ¹⁷⁷Lu, ¹⁸⁶Re, ¹⁸⁸Re, ¹⁰³Pd, ¹⁹⁸Au, ¹⁹²Ir, ⁹⁰Sr, ¹¹¹In or ⁶⁷Ga), which may be covalently bound or non-covalently bound to another species, antineoplastic/antiproliferative/anti-mitotic agents including anti-metabolites such as folic acid analogs/antagonists (e.g., methotrexate, etc.), purine analogs (e.g., 6-mercaptopurine, thioguanine, cladribine, which is a chlorinated purine nucleoside analog, etc.) and pyrimidine analogs (e.g., cytarabine, fluorouracil, etc.), alkaloids including taxanes (e.g., paclitaxel, docetaxel, etc.), alkylating agents such as alkyl sulfonates, nitrogen mustards (e.g., cyclophosphamide, ifosfamide, etc.), nitrosoureas, ethylenimines and methylmelamines, other alkyating agents (e.g., dacarbazine, etc.), antibiotics and analogs (e.g., daunorubicin, doxorubicin, idarubicin, mitomycin, bleomycins, plicamycin, etc.), antiestrogens (e.g., tamoxifen, etc.), antiandrogens (e.g., flutamide, etc.), platinum complexes (e.g., cisplatin, carboplatin, etc.), antineoplastic enzymes (e.g., asparaginase, etc.), agents affecting microtubule dynamics (e.g., vinblastine, vincristine, colchicine, Epo D, epothilone, etc.), caspase activators, proteasome inhibitors, angiogenesis inhibitors (e.g., statins such as endostatin, cerivastatin and angiostatin, squalamine, etc.), etoposides, other agents (e.g., hydroxyurea, flavopiridol, procarbizine, mitoxantrone, campothecin, etc.), various pharmaceutically acceptable salts and derivatives (e.g., esters, etc.) of the foregoing, and combinations of the foregoing, among other agents.

Further therapeutic agents include chemical ablation agents (materials whose inclusion in the formulations of the present invention in effective amounts results in necrosis or shrinkage of nearby tissue upon injection) including osmotic-stress-generating agents (e.g., salts, etc.), basic agents (e.g., sodium hydroxide, potassium hydroxide, etc.), acidic agents (e.g., acetic acid, formic acid, etc.), enzymes (e.g., collagenase, hyaluronidase, pronase, papain, etc.), free-radical generating agents (e.g., hydrogen peroxide, potassium peroxide, etc.), other oxidizing agents (e.g., sodium hypochlorite, etc.), tissue fixing agents (e.g., formaldehyde, acetaldehyde, glutaraldehyde, etc.), coagulants (e.g., gengpin, etc.), non-steroidal anti-inflammatory drugs, contraceptives (e.g., desogestrel, ethinyl estradiol, ethynodiol, ethynodiol diacetate, gestodene, lynestrenol, levonorgestrel, mestranol, medroxyprogesterone, norethindrone, norethynodrel, norgestimate, norgestrel, etc.), GnRH agonists (e.g., buserelin, cetorelix, decapeptyl, deslorelin, dioxalan derivatives, eulexin, ganirelix, gonadorelin hydrochloride, goserelin, goserelin acetate, histrelin, histrelin acetate, leuprolide, leuprolide acetate, leuprorelin, lutrelin, nafarelin, meterelin, triptorelin, etc.), antiprogestogens (e.g., mifepristone, etc.), selective progesterone receptor modulators (SPRMs) (e.g., asoprisnil, etc.), various pharmaceutically acceptable salts and derivatives of the foregoing, and combinations of the foregoing, among other agents.

For tissue bulking applications (e.g., urethral bulking, cosmetic bulking, etc.), specific beneficial therapeutic agents include those that promote collagen production, including proinflammatory agents and sclerosing agents such as those listed Pub. No. US 2006/0251697.

Various procedures have associated with them some degree of pain. Thus, in certain embodiments, the injectable particles of the invention contain one or more agents selected from narcotic analgesics, non-narcotic analgesics, local anesthetic agents and other pain management agents.

Examples of narcotic analgesic agents for use in the present invention may be selected from suitable members of the following: codeine, morphine, fentanyl, meperidine, propoxyphene, levorphanol, oxycodone, oxymorphone, hydromorphone, pentazocine, and methadone, among others, as well as combinations and pharmaceutically acceptable salts, esters and other derivatives of the same.

Examples of non-narcotic analgesic agents for use in the present invention may be selected from suitable members of the following: analgesic agents such as acetaminophen, and non-steroidal anti-inflammatory drugs such as aspirin, diflunisal, salsalate, ibuprofen, ketoprofen, naproxen indomethacin, celecoxib, valdecoxib, diclofenac, etodolac, fenoprofen, flurbiprofen, ketorolac, meclofenamate, meloxicam, nabumetone, naproxen, oxaprozin, piroxicam, sulindac, tolmetin, and valdecoxib, among others, as well as combinations and pharmaceutically acceptable salts, esters and other derivatives of the same.

Examples of local anesthetic agents for use in the present invention may be selected from suitable members of the following: benzocaine, cocaine, lidocaine, mepivacaine, and novacaine, among others, as well as combinations and pharmaceutically acceptable salts, esters and other derivatives of the same.

As noted above, polymeric compositions in accordance with the invention may contain biodegradable ionic polymers with one or more groups that electrostatically interact with a charged therapeutic agent (e.g., a charged radioisotope, a charged small molecule drug, etc.). A benefit of this approach, as it pertains to embolic particles with charged radioisotopes, is that the particles need not be exposed to high energy radiation associated with the conversion of non-radioactive isotopes (e.g., ⁸⁹Y) to radioactive isotopes (e.g., ⁹⁰Y). Instead, the particles can be loaded with the charged radioisotope after it is exposed to the high energy radiation. In this regard, the exposure of many polymers to the levels of radiation needed to convert non-radioactive isotopes to radioactive ones would result in significant changes to the polymers (e.g., extensive chain scission and/or crosslinking) which may alter the chemical and/or mechanical properties of the particles. In this regard, see, e.g., J. F. W. Nijsen et al., Biomaterials 23 (2002) 1831-1839, which resort substantial changes in the molecular weight of polylactic acid upon exposure to radiation.

In some embodiments, particularly those where it is desirable that the polymeric compositions retain charged therapeutic agents such as radioisotopes, among others, it is desirable to further provide the polymeric compositions with species that non-covalently bind to the charged therapeutic agents, for example, based on the formation coordination compounds, complexes, chelates, and so forth. As one specific example, acetyl acetone (2,4-pentanedione) is known to form complexes with charged metal ions including charged radioisotopes. For example, holmium ions are known to form water insoluble complexes with acetyl acetone. In certain embodiments of the invention, polymeric particles in accordance with the invention may first be loaded with acetyl acetone (e.g., concurrent with or after particle formation), followed by subsequent introduction of a radioactive isotope in soluble form (e.g., in the form of a salt solution, for instance, a radioactive metal chloride solution), whereupon the isotope forms an insoluble complex with the acetyl acetone within the particles. The radioisotopes are thus retained, at least temporarily, in the particles.

In certain embodiments, the polymeric compositions of the invention may include one or more imaging agents, for example, radiopaque materials, materials that are visible under magnetic resonance imaging (MRI-visible materials), ferromagnetic materials, and/or ultrasound contrast agents. These materials can, for example, be covalently bonded to non-covalently associated with the polymeric compositions. Various radiopaque materials, MRI-visible materials, ferromagnetic materials, and contrast agents are described, for example, in Pub. No. US 2004/0101564 A1 to Rioux et al.

Polymeric particles for use in the invention may be formed by any suitable particle forming method, including emulsion/solvent evaporation methods, droplet solidification methods, and compression molding methods, among many others.

For example, a droplet forming solution may be formed by dissolving an ionic polymer (e.g., one comprising a biodegradable PTMC core with cationic or anionic end groups), along with optional additional agents such as therapeutic agents and/or ion coordinating agent such as acetyl acetone, among others, in a suitable organic solvent, for example, one capable of dissolving the ionic polymer and the optional additional agents, while also being soluble in a solidification solution as described below. The droplet forming solution may be delivered at a suitable temperature (e.g., temperature can be increased above room temperature to reduce viscosity, as desired) to a drop generator, which forms and directs drops of the solution into a solidification solution, which extracts the organic solvent from the droplets due to the solubility of the organic solvent in the solidification solution, causing them to form solid particles. The particles may then be sorted into desired size ranges. As another example, particles may be formed by not dissolving but rather by “plasticizing” the particle forming material with one or more solvents that disrupt the ionic domains of the material. As yet another example, particles may be formed from a polymer melt and cooled.

To the extent that porous particles are desired, they may be formed by including a material in the particle formation process that is subsequently extracted from the particles. For example, in accordance with an embodiment of the invention, a solution containing an organic solvent, ionic polymer, optional agents such as therapeutic agents or coordinating agents, and a gelling precursor such as sodium alginate may be delivered to drop generator, which forms and directs drops of the solution into a gelling solution containing a gelling agent which interacts with the gelling precursor. For example, in the case where an alginate gelling precursor is employed, an aqueous solution of an agent containing a divalent metal cation, such as calcium chloride, may be used as a gelling agent, which stabilizes the drops by gel formation based on ionic crosslinking (and also possibly by removal of organic solvent as described above). If desired, any residual organic solvent may be allowed to evaporate at this stage. The gel-stabilized drops may then be transferred to a gel dissolution chamber, where the gel is dissolved. For example, ionically crosslinked alginate may be removed by ion exchange with a solution of sodium hexa-metaphosphate. Porosity is generated due to the presence (and ultimate removal) of the alginate. The particles may then be sorted into desired size ranges as above.

Using the above and other techniques, porous particles may be formed having a variety of pore sizes and porosities.

Polymeric coatings in accordance with the invention may be formed by any suitable coating technique, including, for example, contact with a solution or melt that contains the biodegradable ionic polymer and any optional agents (e.g., by dipping, spray coating, coating via an application device, etc.), compression coating based on a powder containing the biodegradable ionic polymer and any optional agents, etc.

Once suitable polymeric compositions (e.g., particles, coatings, etc.) are obtained, in some embodiments, the polymeric compositions may be loaded with a therapeutic agent. In one method, a polymeric composition is exposed to a solution containing one or more therapeutic agents. To increase solution uptake, the polymeric composition may be dried by any suitable method, including lyophilization (freeze drying). In other embodiments, wet polymeric compositions are exposed to the solution Depending on the nature of the therapeutic agents, the solvent systems used to create the solution may be based on (a) water, (b) one or more organic solvents, or (c) water and one or more organic solvents. Typically, the one or more therapeutic agents should be soluble in the selected solvent system. Furthermore, the selected solvent system should not destroy the mechanical integrity of the polymeric compositions.

As noted above, polymeric compositions for use in the invention are formed from polymers that contain ionic end groups, for example, cationic end groups, anionic end groups, or both, and such polymeric compositions may be paired with charged therapeutic agents to take advantage of electrostatic interactions. For example, polymeric compositions having cationic end groups may be paired with negatively charged therapeutic agents, or polymeric compositions having anionic end groups may be paired with positively charged therapeutic agents.

For example, polymeric compositions formed from polymers having anionic end groups may be admixed with a cationic therapeutic agent (e.g., one having one or more —NH₃⁺ groups, ═NH₂⁺ groups, ═NH⁺═ groups, ═N⁺═ groups, etc.), or compositions having formed from polymers having cationic end groups may be admixed with anionic therapeutic agents (e.g., one containing one or more —COO⁻ groups, etc.). Salt forms for cationic charged compositions/agents include those based on inorganic and organic acids (including amino acids, hydroxyacids and fatty acids), for instance, hydrochloride, hydrobromide, sulfate, nitrate, phosphate, mesylate, tosylate, acetate, propionate, maleate, benzoate, salicylate, fumarate, glutamate, aspartate, citrate, lactate, succinate, tartrate, hexanoate, octanoate, decanoate, oleate and stearate salt forms, among others. Salt forms for anionic compositions/agents include those based on alkali/alkaline earth metals and amines (including amino acids), for instance, sodium, potassium, calcium, magnesium, zinc, triethylamine, ethanolamine, triethanolamine, meglumine, ethylene diamine, choline, arginine, lysine and histidine salt forms, among others.

To the extent that the anionic end groups of the polymers within the compositions are in a protonated form (e.g., acidic groups such as —COOH groups, —SO₃H groups, —PO(OH)₂ groups, etc.), they may be admixed with a basic therapeutic agent (e.g., one having one or more basic groups, for instance, —NH₂ groups, ═NH groups, etc.) for loading of the same. In other embodiments, to the extent that the cationic end groups of the polymers within the compositions are in a deprotonated form (e.g., basic groups such as —NH₂ groups, —NHR₁ groups, —NR₁R₂ groups, etc., where and R₁ and R₂ are defined above) basic compositions may be admixed with an acidic therapeutic agent (e.g., one having one or more acidic groups, for instance, (—COOH groups, —SO₃H groups, —PO(OH)₂ groups, etc.). In either case, acid-base neutralization may yield compositions and agents of opposite charge, resulting in electrostatic interactions.

The amount of optional therapeutic agent within the polymeric compositions of the present invention will vary widely depending on a number of factors, including the disease, disorder or condition being treated, the potency of the therapeutic agent, and nature of the implanted device, and for injectable particles, the volume of particles ultimately injected into the subject, among other factors. Typical therapeutic agent concentration ranges are, for example, from about 0.1 or less to 0.2 to 0.5 to 1 to 2 to 5 to 10 to 20 to 50 wt % or more of the therapeutic-agent-containing polymeric compositions, among other specific possibilities.

Injectable polymeric particles in accordance with the invention may be stored and transported in dry form or in wet form (e.g., as an aqueous suspension, so long as hydrolysis does not present a problem). Injectable polymeric particle compositions in accordance with the invention may optionally contain additional agents such as one or more of the following among others: (a) tonicity adjusting agents including sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), (b) suspension agents including various surfactants, wetting agents, and polymers (e.g., albumen, PEO, polyvinyl alcohol, block copolymers, etc.), (c) imaging contrast agents (e.g., Omnipaque™, Visipaque™, etc.), (d) pH adjusting agents including various buffer solutes, and (e) therapeutic agents. Dry or wet compositions may be shipped, for example, in a syringe, catheter, vial, ampoule, or other container. Dry forms may be mixed with an appropriate liquid carrier (e.g. sterile water for injection, physiological saline, phosphate buffer, a solution containing an imaging contrast agent, etc.) prior to administration. In this way the concentration of the composition to be injected may be varied at will, depending on the specific application at hand, as desired by the healthcare practitioner in charge of the procedure. Wet forms (e.g., aqueous suspensions) may also be mixed with a suitable liquid carrier (e.g. sterile water for injection, physiological saline, phosphate buffer, a solution containing contrast agent, etc.) prior to administration, allowing the concentration of administered particles (as well as other optional agents) in the suspension to be reduced prior to injection, if so desired by the healthcare practitioner in charge of the procedure. One or more containers of liquid carrier and/or containers of dissolved therapeutic agent may also be supplied and shipped, along with the dry or wet particles, in the form of a kit.

The amount of injectable particles within a suspension to be injected may be determined by those of ordinary skill in the art. The amount of particles may be limited by the fact that when the amount of particles in the composition is too low, too much liquid may be injected, possibly allowing particles to stray far from the site of injection, which may result in undesired embolization or bulking of vital organs and tissues. When the amount of particles is too great, the delivery device (e.g., catheter, syringe, etc.) may become clogged.

In certain embodiments, the density of the liquid (e.g. aqueous phase) that suspends the particles is close to that of the particles themselves, thereby promoting an even suspension. The density of the aqueous phase may be increased, for example, by increasing the amount of solutes that are dissolved in the aqueous phase, and vice versa.

With respect to compositions, including particles, in accordance with the invention, an “effective amount” may be, for example, (a) an amount sufficient to produce an occlusion or emboli at a desired site in the body, (b) an amount sufficient to achieve the degree of bulking desired (e.g., an amount sufficient to improve urinary incontinence, vesicourethral reflux, fecal incontinence, ISD or gastro-esophageal reflux, or an amount sufficient for aesthetic improvement), or (c) an amount sufficient to locally treat a disease, disorder or condition. Effective doses may also be extrapolated from dose-response curves derived from animal model test systems, among other techniques.

As noted above, permanent or temporary occlusion of blood vessels is useful for managing various diseases, disorders and conditions. Compositions including particles in accordance with the invention may thus be used in the treatment of, for example, fibroids, solid tumors such as renal carcinoma, bone tumor, and cancer of the liver, breast, prostate, lung, thyroid and ovaries, among others, internal bleeding including gastrointestinal, urinary, renal and varicose bleeding, other forms of bleeding including uterine hemorrhage and severe bleeding from the nose (epistaxis), arteriovenous malformations (AVMs) (e.g., abnormal collections of blood vessels, for instance in the brain, which shunt blood from a high pressure artery to a low pressure vein, resulting in hypoxia and malnutrition of those regions from which the blood is diverted), aneurysms such as neurovascular, pulmonary and aortic aneurysms, pulmonary artery pseudoaneurysms, arteriovenous fistulas including intracerebral arteriovenous fistula, cavernous sinus dural arteriovenous fistula and arterioportal fistula, hypervascular tumors, chronic venous insufficiency, varicocele, and pelvic congestion syndrome. The compositions can be used in some embodiments as, for example, fillers for aneurysm sacs, as fillers for AAA sacs (Type II endoleaks), as endoleak sealants, as arterial sealants, or as puncture sealants, and can be used to provide occlusion of other lumens such as fallopian tubes, among many other uses. In some embodiments, a composition containing the particles can be used to prophylactically treat a condition. Moreover, such compositions can be used for preoperative embolization (to reduce the amount of bleeding during a surgical procedure) and occlusion of saphenous vein side branches in a saphenous bypass graft procedure, among other uses. As discussed above, treatment may enhanced in some embodiments of the present invention by the inclusion of one or more therapeutic agents in the particles.

Particles in accordance with the invention may also be used in tissue bulking applications, for example, as augmentative materials in the treatment of urinary incontinence, vesicourethral reflux, fecal incontinence, intrinsic sphincter deficiency (ISD) or gastro-esophageal reflux disease, or as augmentative materials for aesthetic improvement. As above, treatment may be enhanced in the present invention by the presence of one or more therapeutic agent (e.g., proinflammatory agents, sclerosing agents, etc.) in the particles.

The present invention encompasses various ways of administering the particulate compositions of the invention to effect embolization, bulking or other procedure. One skilled in the art can determine the most desirable way of administering the particles depending on the type of treatment and the condition of the patient, among other factors. Methods of administration include, for example, percutaneous techniques as well as other effective routes of administration. For example, the particulate compositions of the invention may be delivered through a syringe or through a catheter, for instance, a FasTracker® microcatheter (Boston Scientific, Natick, Mass., USA), which can be advanced over a guidewire, a steerable microcatheter, or a flow-directed microcatheter (MAGIC, Balt, Montomorency, France).

Various aspects of the invention of the invention relating to the above are enumerated in the following paragraphs:

Aspect 1. Injectable particles comprising a first ionic polymer that comprises a biodegradable polymer core and an ionic end group.

Aspect 2. The injectable particles of Aspect 1, wherein the ionic end group is a cationic end group.

Aspect 3. The injectable particles of Aspect 1, wherein the ionic end group is selected from an —NH₃⁻, —NH₂R₁⁻, —NHR₁R₂⁺, —NR₁R₂ R₃⁺—PH₃⁺, —PH₂R₁⁺, —PHR₁R₂⁺, and —PR₁R₂R₃⁺, where R₁, R₂ and R₃ are independently C1-C10 alkyl.

Aspect 4. The injectable particles of Aspect 1, wherein the ionic end group is an anionic end group.

Aspect 5. The injectable particles of Aspect 1, wherein the ionic end group is selected from —COO⁻, —SO₃⁻, —OSO₃⁻, —PO₂(OH)⁻, —PO₃²⁻, —OPO₂(OH)⁻, and —OPO₃²⁻.

Aspect 6. The injectable particles of Aspect 1, wherein the first ionic polymer comprises a plurality of ionic end groups.

Aspect 7. The injectable particles of Aspect 1, wherein the biodegradable polymer core comprises a biodegradable polymer chain selected from polyester, polycarbonate, poly(ortho ester), polyanhydride, amino-acid-based polycarbonate and amino-acid-based polyester-amide chains.

Aspect 8. The injectable particles of Aspect 1, wherein the biodegradable polymer core is a linear biodegradable polymer core.

Aspect 9. The injectable particles of Aspect 1, wherein the biodegradable polymer core is a branched biodegradable polymer core.

Aspect 10. The injectable particles of Aspect 1, wherein the biodegradable polymer core comprises a biodegradable polymer chain emanating from an initiator molecule residue.

Aspect 11. The injectable particles of Aspect 10, wherein the biodegradable polymer core comprises two to five of the biodegradable polymer chains.

Aspect 12. The injectable particles of Aspect 10, wherein the end of the biodegradable polymer chain comprises an ionic end group.

Aspect 13. The injectable particles of Aspect 10, wherein the end of the biodegradable polymer chain comprises a plurality of ionic end groups.

Aspect 14. The injectable particles of Aspect 1, wherein the injectable particles comprise a mixture of differing first and second ionic polymers, each of which comprises a biodegradable polymer core and an ionic end group.

Aspect 15. The injectable particles of Aspect 14, wherein the first ionic polymer comprises a cationic end group and the second ionic polymer comprises an anionic end group.

Aspect 16. The injectable particles of Aspect 1, further comprising a therapeutic agent.

Aspect 17. The injectable particles of Aspect 14, wherein the therapeutic agent is selected from an anti-tumor agent, a pain relief agent and a sclerosing agent.

Aspect 18. The injectable particles of Aspect 14, wherein the therapeutic agent is radioactive ion.

Aspect 19. The injectable particles of Aspect 18, wherein the radioactive ion is yttrium.

Aspect 20. The injectable particles of Aspect 18, wherein the injectable particles comprise a complexing agent for the radioactive ion.

Aspect 21. The injectable particles of Aspect 18, wherein the complexing agent is acetyl acetate.

Aspect 22. The injectable particles of Aspect 1, wherein 95 vol % of the first and second groups of polymeric particles have a longest linear cross-sectional dimension between 40 μm and 5000 μm.

Aspect 23. The injectable particles of Aspect 1, wherein the particles have a sphericity of 0.8 or more.

Aspect 24. The injectable particles of Aspect 1, wherein the injectable particles are porous.

Aspect 25. The injectable particles of Aspect 1, wherein the particles, when in a dry state, swell by at least 10% within one day of immersion in water.

Aspect 26. A vascular stent comprising a metallic stent substrate and a coating comprising a first ionic polymer that comprises a biodegradable polymer core and an ionic end group.

Although various aspects and embodiments of the invention are specifically illustrated and described herein, it will be appreciated that modifications and variations of the present invention are covered by the above teachings and are within the purview of any appended claims without departing from the spirit and intended scope of the invention.

1. Injectable particles comprising a first ionic polymer that comprises a biodegradable polymer core and an ionic end group.

2. The injectable particles of claim 1, wherein said ionic end group is a cationic end group.

3. The injectable particles of claim 1, wherein said ionic end group is selected from an —NH₃⁺, —NH₂R₁⁺, —NHR₁R₂⁺, —NR₁R₂R₃⁺—PH₃⁺, —PH₂R₁⁺, —PHR₁R₂⁺, and —PR₁R₂ R₃⁺, where R₁, R₂ and R₃ are independently C1-C10 alkyl.

4. The injectable particles of claim 1, wherein said ionic end group is an anionic end group.

5. The injectable particles of claim 1, wherein said ionic end group is selected from —COO⁻, —SO₃⁻, —OSO₃⁻, —PO₂(OH)⁻, —PO₃²⁻, —OPO₂(OH)⁻, and —OPO₃²⁻.

6. The injectable particles of claim 1, wherein said first ionic polymer comprises a plurality of ionic end groups.

7. The injectable particles of claim 1, wherein said biodegradable polymer core comprises a biodegradable polymer chain selected from polyester, polycarbonate, poly(ortho ester), polyanhydride, amino-acid-based polycarbonate and amino-acid-based polyester-amide chains.

8. The injectable particles of claim 1, wherein said biodegradable polymer core is a linear biodegradable polymer core.

9. The injectable particles of claim 1, wherein said biodegradable polymer core is a branched biodegradable polymer core.

10. The injectable particles of claim 1, wherein said biodegradable polymer core comprises a biodegradable polymer chain emanating from an initiator molecule residue.

11. The injectable particles of claim 10, wherein said biodegradable polymer core comprises two to five of the biodegradable polymer chains.

12. The injectable particles of claim 10, wherein said end of the biodegradable polymer chain comprises an ionic end group.

13. The injectable particles of claim 10, wherein said end of the biodegradable polymer chain comprises a plurality of ionic end groups.

14. The injectable particles of claim 1, wherein said injectable particles comprise a mixture of differing first and second ionic polymers, each of which comprises a biodegradable polymer core and an ionic end group.

15. The injectable particles of claim 14, wherein said first ionic polymer comprises a cationic end group and the second ionic polymer comprises an anionic end group.

16. The injectable particles of claim 1, further comprising a therapeutic agent.

17. The injectable particles of claim 14, wherein the therapeutic agent is selected from an anti-tumor agent, a pain relief agent and a sclerosing agent.

18. The injectable particles of claim 14, wherein said therapeutic agent is radioactive ion.

19. The injectable particles of claim 18, wherein said radioactive ion is yttrium.

20. The injectable particles of claim 18, wherein said injectable particles comprise a complexing agent for the radioactive ion.

21. The injectable particles of claim 18, wherein said complexing agent is acetyl acetate.

22. The injectable particles of claim 1, wherein 95 vol % of said first and second groups of polymeric particles have a longest linear cross-sectional dimension between 40 μm and 5000 μm.

23. The injectable particles of claim 1, wherein said particles have a sphericity of 0.8 or more.

24. The injectable particles of claim 1, wherein said injectable particles are porous.

25. The injectable particles of claim 1, wherein said particles, when in a dry state, swell by at least 10% within one day of immersion in water.

26. A vascular stent comprising a metallic stent substrate and a coating comprising a first ionic polymer that comprises a biodegradable polymer core and an ionic end group.
